(19) United States
(12) Patent Application Publication
(10) Pub. No.: US 2015/0157198 A1
(43) Pub. Date: Jun. 11, 2015

(54) Ophthalmic Illumination System with Micro-Display Overlaid Image Source

(71) Applicant: Topcon Medical Laser Systems, Inc., Santa Clara, CA (US)

(72) Inventors: Chris Sramek, Sunnyvale, CA (US); Greg Halstead, Sunnyvale, CA (US)

(73) Assignee: Topcon Medical Laser Systems, Inc., Santa Clara, CA (US)

(21) Appl. No.: 14/103,468

(22) Filed: Dec. 11, 2013

(51) Int. Cl. A61B 3/00

(52) U.S. Cl. CPC A61B 3/0008

(57) ABSTRACT

Methods and apparatuses for an ophthalmic imaging system are provided. An ophthalmic illumination system with a micro-display projector based head-up display is provided. In accordance with an embodiment, a first optical element is configured to direct light from a light source upon an eye to be examined. A micro-display projector is configured to generate a micro-display image including information associated with the eye to be examined. A third optical element is configured to receive reflected light from the eye resulting from the light directed upon the eye, receive the micro-display image, and transmit at least a portion of the reflected light and at least a portion of light from the micro-display image.

### TECHNICAL FIELD

The present disclosure is generally directed to ophthalmic illumination systems for use in diagnosing and treating conditions of the eye, and more specifically to systems and methods for generating overlaid images for use in combination with ophthalmic illumination systems.

### BACKGROUND

A conventional slit lamp is an instrument consisting of a high-intensity light source. The high-intensity light source can be focused to shine a beam of light into a patient's eye. The beam of light is often focused to shine a desired light pattern into the patient's eye, such as a thin slit-shaped sheet of light.

Slit lamps are typically used in ophthalmic illumination systems to allow a practitioner to diagnose and treat conditions of the eye, e.g., by enabling a practitioner to view the patient's eye. For example, a slit lamp may be a component of a clinical bio-microscope used to facilitate an examination of structures within a patient's eye, including the eyelid, retina, sclera, conjunctiva, iris, lens and cornea.

Many existing ophthalmic illumination systems allow a practitioner to view an image of a patient's eye and overlay additional information onto the image to enhance the image or for the practitioner's convenience. For example, overlaid information may indicate one or more regions targeted for treatment, guidance relevant to a treatment region, or other useful information.

In order to not compromise the image of the primary source (for example, the patient's eye), many existing systems combine a large quantity of light associated with the primary source with a relatively smaller quantity of light associated with the overlaid information. In many existing ophthalmic illumination systems, limiting the quantity of light associated with the overlaid information reduces the quality of the resulting composite image.

### SUMMARY

An ophthalmic illumination system with a micro-display overlaid image source is provided. In accordance with an embodiment, a first optical element is configured to direct light from a light source upon an eye to be examined. A micro-display projector is configured to generate a micro-display image including information associated with the eye to be examined. A third optical element is configured to receive reflected light from the eye resulting from the light directed upon the eye, receive the micro-display image, and transmit at least a portion of the reflected light and at least a portion of the micro-display image. The micro-display projector may include one of a liquid crystal on silicon (LCoS), digital-micro-mirror (DMD) or micro-electro-mechanical systems (MEMS) micro-scanner, and one of a light-emitting diode (LED) or red-green-blue (RGB) laser light source.

In accordance with an embodiment, the third optical element may be further configured to transmit a stereoscopic image of the portion of the reflected light and the portion of the micro-display image. The third optical element may be a beam-splitter.

In accordance with an embodiment, a controller may be configured to receive a parameter for generating the micro-display image, and transmit a command based on the parameter to the micro-display projector.

In accordance with an embodiment, the light from the light source may define an illuminated area, the illuminated area being one of a slit-shaped, round or polygonal-shaped area.

In accordance with an embodiment, the micro-display image may relate to measurement information, patient data, a treatment parameter, a preoperative image, a treatment plan, an aiming beam pattern or a treatment beam target indicator.

These and other advantages of the invention will be apparent to those of ordinary skill in the art by reference to the following detailed description and the accompanying drawings.

### BRIEF DESCRIPTION OF THE DRAWINGS

FIG. 1 shows a prior art ophthalmic illumination system with a conventional overlaid image source;

FIG. 2 shows an ophthalmic illumination system with a micro-display overlaid image source in accordance with an embodiment;

FIG. 3 shows a composite image of a patient's eye generated by an ophthalmic illumination system with a micro-display overlaid image source in accordance with an embodiment;

FIG. 4 shows a stereoscopic ophthalmic illumination system with a micro-display overlaid image source in accordance with an embodiment;

FIG. 5 is a flowchart of an ophthalmic illumination method in accordance with an embodiment; and

FIG. 6 is a high-level block diagram of an exemplary computer that may be used for the various embodiments herein.

### DETAILED DESCRIPTION

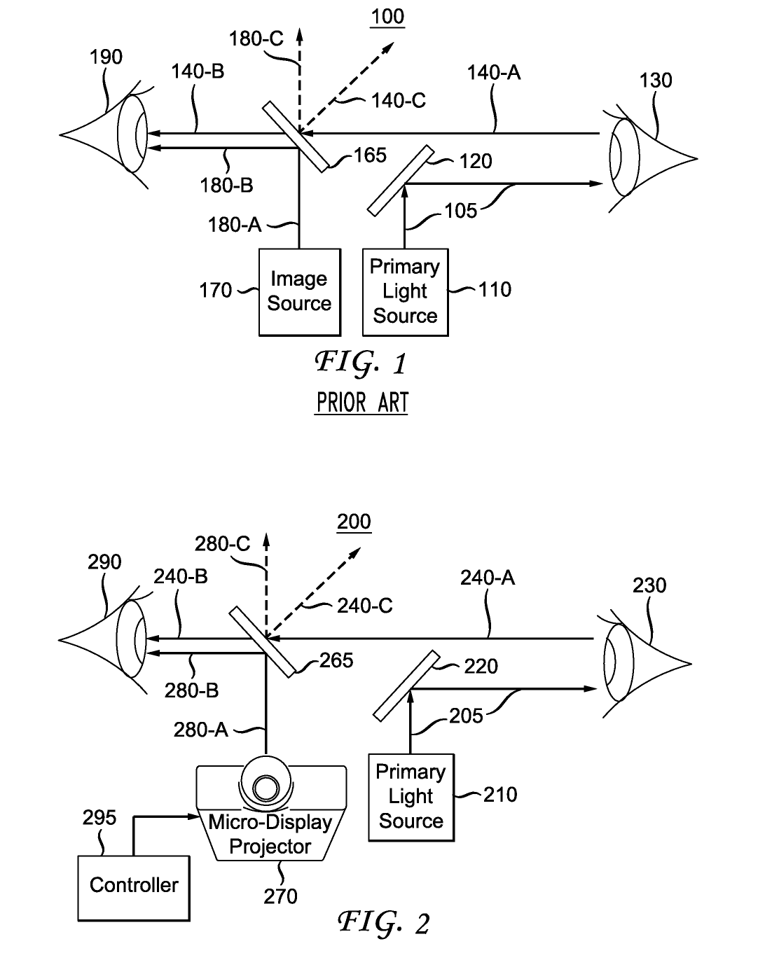

FIG. 1 shows a prior art ophthalmic illumination system with a conventional overlaid image source. Imaging system 100 comprises primary light source 110, mirror 120, image source 170 and beam-splitter 165. Primary light source 110 may comprise a slit lamp that includes a high-intensity/high-pressure light source, such as a halogen light source that produces and channels light to various elements (not shown), including a slit adjustment mechanism, optical relay, filter wheel, slit rotation prism assembly and exit (turning) prism/mirror.

In a conventional imaging system, primary light source 110 generates light 105 which is directed by mirror 120 toward patient's eye 130. The light strikes patient's eye 130 and is reflected, generating reflected light 140-A. Reflected light 140-B passes through beam-splitter 165 and propagates toward practitioner's eye 190, allowing a practitioner to view structures within patient's eye 130. Beam splitter 165 is typically adapted to allow a significant amount of reflected light 140-A from patient's eye 130 to pass through depending on the application, although some amount of the reflected light, shown as 140-C, is lost.

Image source 170 generates an image to be overlaid onto the image of patient's eye 130. Illustratively, image source 170 transmits the image via light 180-A. Light 180-A is directed toward beam-splitter 165, and reflected light 180-B is transmitted by beam-splitter 165 toward practitioner's eye 190. Typically, a significant portion of light from image source 170, shown as 180-C, is intentionally lost to create an overlaid image effect. Practitioner's eye 190 accordingly receives both light 140 and light 180-B, and views a composite image that includes an image of patient's eye 130 and the overlaid image generated by image source 170.

It is known that the use of a beam-splitter is associated with a trade-off: if more of the light from the primary source (i.e., light 140) is allowed to pass through the beam-splitter, more of the light from the image source (i.e., light 180-A) is blocked. Because practitioners typically require a clear image of a patient's eye to be examined, existing systems typically permit a relatively large portion of the light from the primary source to pass through the beam-splitter. As a result, in many existing illumination systems the quality of the overlaid image generated by the image source is reduced. Accordingly, there is a need for illumination systems which produce a composite image that includes a relatively large portion of reflected light from a patient's eye (reflected as a result of light from the primary light source being directed upon the eye) and a high quality version of the overlaid image generated by the image source. In the embodiments herein, a micro-display overlaid image source (e.g., a micro-display projector) is employed to generate high-quality overlaid images relative to those of a typical conventional image source. The lower amounts of light necessary for high-quality overlaid micro-display images can improve a practitioner's view of both structures within a patient's eye and overlaid information.

FIG. 2 shows an ophthalmic illumination system with a micro-display overlaid image source in accordance with an embodiment. Illumination system 200 comprises primary light source 210, mirror 220, micro-display projector 270 and beam splitter 265. Primary light source 210 generates light 205 which is directed by mirror 220 toward patient's eye 230. Light 205 strikes patient's eye 230 and is reflected, generating reflected light 240-A. Reflected light 240-B passes through beam-splitter 265 and propagates toward practitioner's eye 290, allowing the practitioner to view structures within patient's eye 230.

Micro-display projector 270 generates a micro-display image to be overlaid onto the image of patient's eye 230. In an illustrative embodiment, micro-display projector 270 transmits a micro-display image via light 280-A. Light 280-A is received by beam-splitter 265, and a portion of the light 280-B is transmitted by beam-splitter 265 toward practitioner's eye 290. Practitioner's eye 290 accordingly receives both light 240-B and light 280-B, and views a composite image that includes an image of patient's eye 230 and the overlaid image generated by micro-display projector 270.

In accordance with an embodiment, beam-splitter 265 is configured to transmit a composite image in which preferrably about five to fifteen percent (5-15%) of light (as shown by light 280-B) associated with the overlaid micro-display image is transmitted (i.e., reflected), resulting in a high-quality overlaid image. For example, about ninety percent (90%) of light 280-C associated with the overlaid micro-display image passes through beam splitter 265 and is lost when beam-splitter 265 is configured to transmit about ten percent (10%) of the overlaid micro-display image. However, one skilled in the art will appreciate that other ratios of projected light allowed to pass through and projected light to be transmitted are possible. Further, in an embodiment beam-splitter 265 is configured to preferably allow between about ninety to ninety-nine (90-99%) of the reflected light, shown as 240-B, from patient's eye 230 to pass through toward practitioner's eye 290. For example, between about one percent (1%) of the reflected light, shown as 240-C, is reflected by beam-splitter 265 and is lost when beam-splitter 265 is configured to allow ninety-nine (99%) of the reflected light from patient's eye 230 to pass through toward practitioner's eye 290. Again, one skilled in the art will appreciate that other ratios of reflected light 240-A allowed to pass through or be reflected are possible.

Beam-splitter 265 may be any type of beam-splitter configured to perform the embodiments herein. For example, beam-splitter 265 may comprise a glass or plastic cube, a half-silvered mirror (e.g., a sheet of glass or plastic with a thin coating of metal or dichroic optical coating) or a dichroic mirrored prism.

Micro-display projector 270 may be any type of micro-display or pico projector comprising an optical engine (e.g., an illumination source, modulator and projection optics). For example, micro-display projector 270 may be a stand-alone projector or a projector that is integrated into another device, such as a mobile device (e.g., a mobile phone) or a notebook computer.

Micro-display projector 270 may include one of a liquid crystal on silicon (LCoS), digital-micro-mirror device (DMD), 2-D micro-electro-mechanical systems (MEMS) or 2-D X/Y galvanometer set micro-scanner for generating an image. Micro-display projector 270 also may comprise relay optics (e.g., to illuminate a micro-display with an illumination area dimension matching the micro-display size), and a collimation or projection lens.

Further, micro-display projector 270 may include one or more sources of visible and/or invisible illumination to be operable to form, e.g., an infrared or color image projection. The one or more sources of visible and/or invisible illumination may include a halogen lamp, a white light emitting diode (LED), one or more coaxial LEDs (e.g., red, green, blue, amber or near-infrared LEDs) or one or more coaxial lasers (e.g., red-green-blue (RGB) or near-infrared lasers). In an embodiment, an exemplary light source for micro-display projector 270 may have an illumination range of around 10-200 lumens. One skilled in the art will note that micro-display projector 270 may include several other elements, and that the micro-display projector features and components discussed herein are merely illustrative and, therefore, are not intended to be exhaustive.

In an embodiment, controller 295 may be configured to receive user inputs via control switches, knobs, or a GUI interface (e.g. a touch-screen display or LCD with a mouse/trackpad interface), and transmit one or more commands to micro-display projector 270 to generate a micro-display projection 280-A based on the one or more received user inputs. Controller 295 also may transmit one or more commands to micro-display projector 270 to adjust the color, brightness and timing of micro-display projection 280-A based on one or more user inputs. Controller 295 also may be configured to receive inputs from one or more external sources (e.g. a camera flash trigger or a computer processing real-time slit-lamp video) and transmit commands to micro-display projector 270.

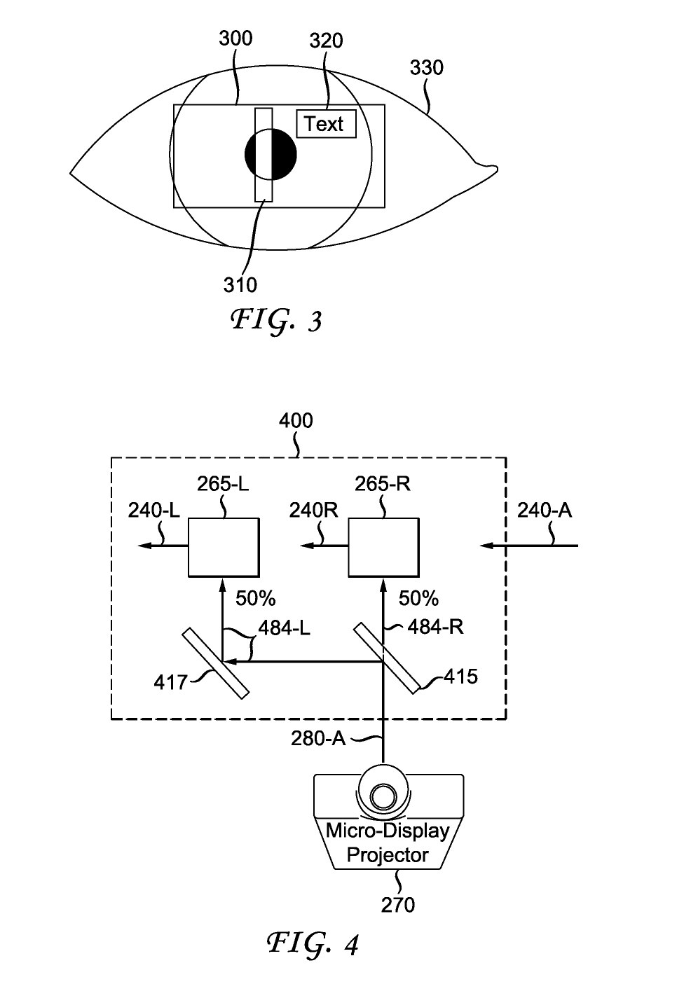

FIG. 3 shows a composite image of a patient's eye generated by an ophthalmic illumination system with a micro-display overlaid image source in accordance with an embodiment. Composite image 300 includes an image of patient's eye 330, received after light from light source 210 is directed by mirror 220 (shown in FIG. 2) onto patient's eye 330, and visual information generated by micro-display projector 270. In an embodiment, micro-display projector 270 generates concurrent information 320 that is overlaid to form composite image 300. Alternatively, all or part of concurrent information 320 may be received from a source external to micro-display projector 270 (e.g., from controller 295, or a source other than controller 295).

For example, concurrent information 320 may include visual information received or generated by micro-display projector 270, including any type of image or data that may be associated with patient's eye 330. Concurrent information 320 may include patient information, the current time and date, or other information that may be of use in a clinical environment. In another example, concurrent information 320 may include measurement information, such as a measurement axis, distance, area, scale or grid. Measurement information also may include a current illumination area diameter, current slit width, inter-slit spacing, current filter choice, micrometer scale labeling, or circle/ellipse radii, ratios and areas.

When illumination system 200 is used in conjunction with therapy systems including laser systems and other equipment, concurrent information 320 may include one of a treatment parameter or a preoperative image, treatment plan, an aiming beam pattern or a treatment beam target indicator. For example, concurrent information 320 may be received from a laser system console to include information regarding treatment laser parameters, such as, e.g., power, spot-size and spacing.

FIG. 4 shows a stereoscopic ophthalmic illumination system with a micro-display overlaid image source in accordance with an embodiment. Stereoscopic system 400 includes a beam splitter 415, a mirror 417, a beam-splitter 265-L and a beam splitter 265-R. Stereoscopic imaging system 400 may be combined with illumination system 200 to provide stereo imaging, for example, by providing a separate image for each of a practitioner's eyes.

Referring to FIGS. 2 and 4, beam-splitter 265 is replaced by stereoscopic imaging system 400 to provide stereo imaging. In an illustrative embodiment, reflected light 240-A from patient's eye 230 passes through both beam-splitters 265-L, 265-R, and propagates toward practitioner's eye 290, in a manner similar to that described above. Reflected light 240-A may be split by a beam-splitter (not shown), for example, to produce separate beams for beam-splitters 265-L, 265-R. As such, reflected light 240-L and 240-R are the stereoscopic equivalent of the reflected light, shown as 240-B in FIG. 2, allowed to pass through toward practitioner's eye 290.

Micro-display projector 270 projects light 280-A representing an image to be overlaid onto an image represented by light 240-A, in a manner similar to that described above. A first portion 484-R of light 280-A passes through beam-splitter 415 toward beam-splitter 265-R. A second portion 484-L of light 280-A is reflected by beam-splitter 415 toward mirror 417. Light 484-L is reflected by mirror 417 toward beam-splitter 265-L. In one embodiment, portion 484-L comprises fifty percent (50%) of light 280-A, and portion 484-R comprises fifty percent (50%) of light 280-A.

Each beam-splitter 265-L, 265-R functions in a manner similar to beam-splitter 265 described above.

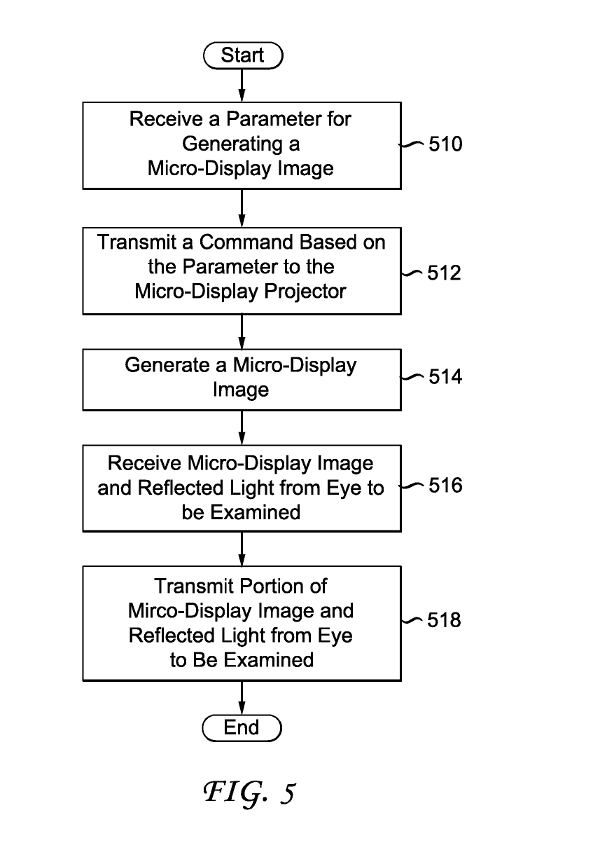

FIG. 5 is a flowchart of an ophthalmic illumination method in accordance with an embodiment. FIG. 5 is discussed below with reference also to FIG. 2.

At step 510, a parameter for generating the micro-display image is received. Referring to FIG. 2, controller 295 may be configured to receive a parameter for generating the micro-display image, wherein the parameter is related to concurrent information relating to patient data, a treatment parameter, a preoperative image, or a treatment plan.

At step 512, a command based on the parameter is transmitted to micro-display projector 270. Referring to FIG. 2, controller 295 transmits a command based on the parameter to micro-display projector 270, wherein micro-display projector 270 generates micro-display image projection 280-A in accordance with the command.

At step 514, micro-display projector 270 is configured to generate a micro-display image. For example, micro-display projector 270 may be a micro-display projector including one of a liquid crystal on silicon (LCoS), digital-micro-mirror (DMD) or micro-electro-mechanical systems (MEMS) micro-scanner and one of a light-emitting diode (LED) or red-green-blue (RGB) laser light source. Referring to FIG. 2, micro-display projector 270 generates micro-display image 280-A (e.g., in accordance with the command received from controller 295). For example, micro-display image 280-A may be related to concurrent information relating to patient data, a treatment parameter, a preoperative image, or a treatment plan.

At step 516, a third optical element is configured to receive the micro-display image 280-A and reflected light 240 from patient's eye 230. Referring to FIG. 2, mirror 220 directs light 205 generated by light source 210 toward a patient's eye 230. Light 205 is reflected by eye 230, generating reflected light 240 which propagates toward a beam-splitter 265. For example, the reflected light may include an image of structures within patient's eye 230 due to an illuminated area of light generated by light source 210.

At step 518, the third optical element is configured to transmit at least a portion of the reflected light and a portion of light from the micro-display image toward physician's eye 290 for examination. Referring to FIG. 2, light 280-A is received by beam-splitter 265, and a portion of the light 280-B is transmitted by beam-splitter 265 toward practitioner's eye 290. Practitioner's eye 290 accordingly receives both reflected light 240 and light 280-B, and views a composite image that includes an image of patient's eye 230 and the overlaid image generated by micro-display projector 270. For example, beam-splitter 265 may be configured to transmit about ten percent (10%) of light 280-B and allow about ninety percent (90%) of light 280-A to pass through (to be lost). Beam-splitter 265 also may be configured to allow about ninety-nine (99%) of reflected light 240 to pass through toward practitioner's eye 290 and allow about one percent (1%) of light 240 to be reflected and lost.

As such, a slit-lamp illumination system with a micro-display overlaid image source as disclosed herein may serve as a replacement for a slit-lamp illuminator with a traditional overlaid image source.

Systems, apparatus, and methods described herein may be implemented using digital circuitry, or using one or more computers using well-known computer processors, memory units, storage devices, computer software, and other components. Typically, a computer includes a processor for executing instructions and one or more memories for storing instructions and data. A computer may also include, or be coupled to, one or more mass storage devices, such as one or more magnetic disks, internal hard disks and removable disks, magneto-optical disks, optical disks, etc.

Systems, apparatus, and methods described herein may be implemented using computers operating in a client-server relationship. Typically, in such a system, the client computers are located remotely from the server computer and interact via a network. The client-server relationship may be defined and controlled by computer programs running on the respective client and server computers.

Systems, apparatus, and methods described herein may be used within a network-based cloud computing system. In such a network-based cloud computing system, a server or another processor that is connected to a network communicates with one or more client computers via a network. A client computer may communicate with the server via a network browser application residing and operating on the client computer, for example. A client computer may store data on the server and access the data via the network. A client computer may transmit requests for data, or requests for online services, to the server via the network. The server may perform requested services and provide data to the client computer(s). The server may also transmit data adapted to cause a client computer to perform a specified function, e.g., to perform a calculation, to display specified data on a screen, etc. For example, the server may transmit a request adapted to cause a client computer to perform one or more of the method steps described herein, including one or more of the steps of FIG. 5. Certain steps of the methods described herein, including one or more of the steps of FIG. 5, may be performed by a server or by another processor in a network-based cloud-computing system. Certain steps of the methods described herein, including step 512 of FIG. 5, may be performed by a client computer in a network-based cloud computing system. The steps of the methods described herein, including step 512 of FIG. 5, may be performed by a server and/or by a client computer in a network-based cloud computing system, in any combination.

Systems, apparatus, and methods described herein may be implemented using a computer program product tangibly embodied in an information carrier, e.g., in a non-transitory machine-readable storage device, for execution by a programmable processor; and the method steps described herein, including one or more of the steps of FIG. 5, may be implemented using one or more computer programs that are executable by such a processor. A computer program is a set of computer program instructions that can be used, directly or indirectly, in a computer to perform a certain activity or bring about a certain result. A computer program can be written in any form of programming language, including compiled or interpreted languages, and it can be deployed in any form, including as a stand-alone program or as a module, component, subroutine, or other unit suitable for use in a computing environment.

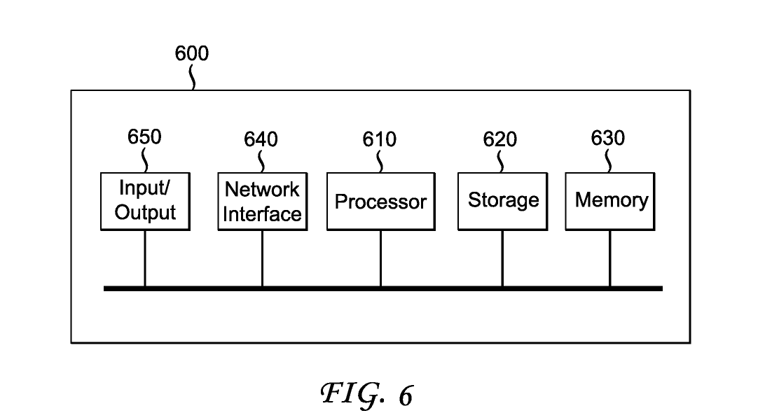

A high-level block diagram of an exemplary computer that may be used to implement systems, apparatus and methods described herein is illustrated in FIG. 6. Computer 600 comprises a processor 610 operatively coupled to a data storage device 620 and a memory 630. Processor 610 controls the overall operation of computer 600 by executing computer program instructions that define such operations. The computer program instructions may be stored in data storage device 620, or other computer readable medium, and loaded into memory 630 when execution of the computer program instructions is desired. Thus, the method steps of FIG. 5 can be defined by the computer program instructions stored in memory 630 and/or data storage device 620 and controlled by processor 610 executing the computer program instructions. For example, the computer program instructions can be implemented as computer executable code programmed by one skilled in the art to perform an algorithm defined by the method steps of FIG. 5. Accordingly, by executing the computer program instructions, the processor 610 executes an algorithm defined by the method steps of FIG. 5. Computer 600 also includes one or more network interfaces 640 for communicating with other devices via a network. Computer 600 also includes one or more input/output devices 650 that enable user interaction with computer 600 (e.g., display, keyboard, mouse, speakers, buttons, etc.).

Processor 610 may include both general and special purpose microprocessors, and may be the sole processor or one of multiple processors of computer 600. Processor 610 may comprise one or more central processing units (CPUs), for example. Processor 610, data storage device 620, and/or memory 630 may include, be supplemented by, or incorporated in, one or more application-specific integrated circuits (ASICs) and/or one or more field programmable gate arrays (FPGAs).

Data storage device 620 and memory 630 each comprise a tangible non-transitory computer readable storage medium. Data storage device 620, and memory 630, may each include high-speed random access memory, such as dynamic random access memory (DRAM), static random access memory (SRAM), double data rate synchronous dynamic random access memory (DDR RAM), or other random access solid state memory devices, and may include non-volatile memory, such as one or more magnetic disk storage devices such as internal hard disks and removable disks, magneto-optical disk storage devices, optical disk storage devices, flash memory devices, semiconductor memory devices, such as erasable programmable read-only memory (EPROM), electrically erasable programmable read-only memory (EEPROM), compact disc read-only memory (CD-ROM), digital versatile disc read-only memory (DVD-ROM) disks, or other non-volatile solid state storage devices.

Input/output devices 650 may include peripherals, such as a printer, scanner, display screen, etc. For example, input/output devices 650 may include a display device such as a cathode ray tube (CRT), plasma or liquid crystal display (LCD) monitor for displaying information to the user, a keyboard, and a pointing device such as a mouse or a trackball by which the user can provide input to computer 600.

Any or all of the systems and apparatus discussed herein, including micro-display projector 210 and controller 295 may be implemented using a computer such as computer 600.

One skilled in the art will recognize that an implementation of an actual computer or computer system may have other structures and may contain other components as well, and that FIG. 6 is a high level representation of some of the components of such a computer for illustrative purposes.

The foregoing Detailed Description is to be understood as being in every respect illustrative and exemplary, but not restrictive, and the scope of the invention disclosed herein is not to be determined from the Detailed Description, but rather from the claims as interpreted according to the full breadth permitted by the patent laws. It is to be understood that the embodiments shown and described herein are only illustrative of the principles of the present invention and that various modifications may be implemented by those skilled in the art without departing from the scope and spirit of the invention. Those skilled in the art could implement various other feature combinations without departing from the scope and spirit of the invention.

We claim:

1. An ophthalmic illumination system, comprising:
a first optical element configured to direct light from a light source upon an eye to be examined;
a micro-display projector configured to generate a micro-display image including information associated with the eye to be examined; and
a third optical element configured to:
receive reflected light from the eye resulting from the light directed upon the eye;
receive the micro-display image; and
transmit at least a portion of the reflected light and at least a portion of light from the micro-display image.

2. The system of claim 1, wherein the third optical element is further configured to transmit a stereoscopic image of the portion of the reflected light and the portion of light from the micro-display image.

3. The system of claim 1, wherein the third optical element is a beam-splitter.

4. The system of claim 1, further comprising a controller configured to:
receive a parameter for generating the micro-display image; and
transmit a command based on the parameter to the micro-display projector.

5. The system of claim 1, wherein the light from the light source defines an illuminated area, the illuminated area being one of a slit-shaped, round or polygonal-shaped area.

6. The system of claim 1, wherein the micro-display image relates to measurement information, patient data, a treatment parameter, a preoperative image, a treatment plan, an aiming beam pattern or a treatment beam target indicator.

7. The system of claim 1, wherein the micro-display projector includes one of a liquid crystal on silicon (LCoS), digital-micro-mirror (DMD) or micro-electro-mechanical systems (MEMS) micro-scanner.

8. The system of claim 1, wherein the micro-display projector includes one of a light-emitting diode (LED) or red-green-blue (RGB) laser light source.

9. The system of claim 1, wherein the portion of the reflected light is about 99 percent.

10. The system of claim 1, wherein the portion of light from the micro-display image is about 10 percent.

11. An ophthalmic illumination method, comprising:
directing light from a light source upon an eye to be examined;
receiving reflected light from the eye resulting from the light directed upon the eye;
generating a micro-display image including information associated with the eye to be examined; and
transmitting at least a portion of reflected light from the eye resulting from of the light directed from the light source and at least a portion of light from the micro-display image.

12. The method of claim 11, further comprising transmitting a stereoscopic image of the portion of the light reflected and the portion of light from the micro-display image.

13. The method of claim 11, further comprising:
receiving a parameter for generating the micro-display image; and
transmitting a command based on the parameter.

14. The method of claim 11, wherein the light from the light source defines an illuminated area, the illuminated area being one of a slit-shaped, round or polygonal-shaped area.

15. The method of claim 11, wherein the micro-display image relates to measurement information, patient data, a treatment parameter, a preoperative image, a treatment plan, an aiming beam pattern or a treatment beam target indicator.

16. The method of claim 11, wherein the portion of the reflected light is about 99 percent.

17. The method of claim 11, wherein the portion of light from the micro-display image is about 10 percent.

18. An ophthalmic illumination system, comprising:
a slit lamp configured to generate light defining an illuminated area;
a mirror configured to direct the light from the slit lamp upon an eye to be examined;
a micro-display projector configured to generate a micro-display image including information associated with the eye to be examined; and
a beam-splitter configured to:
receive reflected light from the eye resulting from the light directed upon the eye;
receive the micro-display image; and
transmit at least a portion of the reflected light and at least a portion of light from the micro-display image.

19. The system of claim 18, wherein the beam-splitter is further configured to transmit a stereoscopic image of the portion of the reflected light and the portion of light from the micro-display image.

20. The system of claim 18, further comprising a controller configured to:
receive a parameter for generating the micro-display image; and
transmit a command based on the parameter to the micro-display projector.

21. The system of claim 18, wherein the illuminated area is one of a slit-shaped, round or polygonal-shaped area.

22. The system of claim 18, wherein the micro-display image relates to measurement information, patient data, a treatment parameter, a preoperative image, a treatment plan, an aiming beam pattern or a treatment beam target indicator.

23. The system of claim 18, wherein the micro-display projector includes one of a liquid crystal on silicon (LCoS), digital-micro-mirror (DMD) or micro-electro-mechanical systems (MEMS) micro-scanner.

24. The system of claim 18, wherein the micro-display projector includes one of a light-emitting diode (LED) or red-green-blue (RGB) laser light source.

25. The system of claim 18, wherein the portion of the reflected light is about 90-99 percent.

26. The system of claim 18, wherein the portion of light from the micro-display image is about 5-15 percent.
